(19) United States
(12) Patent Application Publication
(10) Pub. No.: US 2002/0142297 A1
(43) Pub. Date: Oct. 3, 2002

(54) Compositions and methods for imaging gene expression

(75) Inventors: Alexei A. Bogdanov, Arlington, MA (US); Ralph Weissleder, Charlestown, MA (US); Maria Simonova, Boston, MA (US)

Correspondence Address: J. PETER FASSE, Fish & Richardson P.C., 225 Franklin Street, Boston, MA, 02110-2804, US

(73) Assignee: The General Hospital Corporation, a Massachusetts corporation

(21) Appl. No.: 09/739,068

(22) Filed: Dec. 18, 2000

Related U.S. Application Data
(63) Continuation of application No. 09/015,366, filed on Jan. 29, 1998, now abandoned.
(60) Provisional application No. 60/037,350, filed on Jan. 31, 1997.

(51) Int. Cl. C12Q 1/68; G01N 33/53; C07K 14/435

(52) U.S. Cl. 435/6; 435/7.1; 530/400

(57) ABSTRACT

Described are short peptide sequences, termed recombinant peptide chelates (RPCs), and the imaging marker genes that encode them. The RPCs can be expressed in parallel with the expression of any other desired gene (e.g., a therapeutic gene), and used to easily confirm the expression of the therapeutic gene product. The RPCs are expressed in the cell or on the cell surface concurrently with the therapeutic gene product, and can be assayed by standard imaging techniques.

### CROSS REFERENCE TO RELATED APPLICATIONS

This application claims priority from provisional application 60/037,350, filed Jan. 31, 1997.

### BACKGROUND OF THE INVENTION

The invention relates to the synthesis and use of imaging marker genes (IMGs) encoding recombinant peptide chelates (RPCs), that bind certain metal ions and metal-containing compounds, to image the expression of gene products.

Current methods of measuring gene expression include: 1) tissue sampling to directly determine the presence of expressed protein; and 2) quantitation of a marker gene product in circulation. Both methods are invasive, and only the former method provides anatomical (i.e., localized) data of in vivo gene expression.

Non-invasive approaches to in vivo gene expression imaging that allow precise localization of the expression site and quantitative assessment of the gene expression levels are highly desirable for evaluation of gene therapy trials. One strategy includes radionuclide imaging of heroes simplex virus 1 thymidine kinase (HSV1-tk) marker gene expression using radioiodinated substrate analogs (Iwashima et al., Drug Design and Delivery, 3:309-321, 1988), either in tumor cells in vitro (Tjuvajev et al., Cancer Res., 55:6126-6132, 1995), or after direct injection with recombinant STK retrovirus (Tjuvajev et al., Cancer Res., 56:4087-4095, 1996). A second strategy employs nuclear magnetic resonance (NMR) imaging for detection of transferrin gene overexpression in vivo. The latter method is based on the ability to detect changes in proton relaxation time in cells having excess transferrin-associated paramagnetic iron (Koretsky, A. P. et al. Proceedings of the 4th International Society of Magnetic Resonance in Medicine, p. 69; 1996). The size of HSV Tc and transferrin receptor genes is close to the limit for the amount of DNA that can be reliably introduced into an expression vector, and therefore can restrict the size of any therapeutic gene that can be inserted in the same expression vector.

### SUMMARY OF THE INVENTION

The invention is based on the discovery that short peptide sequences, termed recombinant peptide chelates (RPCs), can be expressed in parallel with the expression of any other desired gene (e.g., a therapeutic gene) inserted into the same vector, and used to easily confirm the expression of the therapeutic gene product. The RPCs provide a qualitative as well as a semiquantitative image of exactly where and to what extent the desired gene is expressed. The genes encoding the RPCs are called imaging marker genes (IMGs) and can be inserted into any vector alongside a separate, desired gene.

The RPCs are expressed in the cell or on the cell surface concurrently with the expression of the therapeutic (or other) gene product. The expressed RPCs can be attached to the outer surface plasma membrane on the cell or can be secreted by the cell into the extracellular space immediately outside of the cell. The RPCs can be detected non-invasively by systemic administration (e.g., by intra-arterial, intravenous, or direct injection) of a metal compound (e.g., ^{99m}Tc(V)O, Re(V), ¹¹¹In, ¹¹³In, or ⁶⁷Ga ion, or a lanthanide paramagnetic metal ion or complex), which forms a thermodynamically and kinetically stable complex with the RPCs on the cell surface or in the extracellular space.

This complexation results in localization of the metal in close proximity to the cells that express the RPC, which in turn allows spatial localization of the specific site of therapeutic gene expression, using standard imaging methods (e.g., radionuclide imaging or NMR).

In general, the invention features a recombinant peptide chelate including the structure:

(XGₐC)_{b}Z_{c}CG_{d}X;

wherein X and Z are any amino acid, e.g., each Z, if present, can be selected independently from the group consisting of valine, proline, and glycine; G is glycine; C is cysteine; a is 1, 2, 3, or 4; b is 1 or 2; c is absent or 1 to 4; and d is 1, 2, 3, or 4. The structure can be repeated one or more times in the same molecule, linked by, e.g., peptide bonds. The invention also features an imaging marker gene including a nucleic acid sequence that encodes a recombinant peptide chelate having this structure.

In another aspect, the invention features a method of monitoring gene expression of a polypeptide, e.g., a therapeutic polypeptide, in a host by introducing into the host an expression vector including a nucleic acid sequence encoding a therapeutic polypeptide and an imaging marker gene (IMG) encoding a recombinant peptide chelate (RPC) which chelates a metal compound; administering to the host the metal compound, e.g., a radioisotope, chelated by the RPC in an amount sufficient to form RPC-metal complexes in the host; and assaying for the RPC-metal complexes as an indication of expression of the therapeutic polypeptide. The expression vector can be prepared by obtaining a nucleic acid sequence encoding a therapeutic polypeptide; obtaining an imaging marker gene (IMG) encoding a recombinant peptide chelate (RPC), e.g., having the structure defined above, which chelates a metal compound; and inserting the IMG and the nucleic acid sequence into an expression vector.

The metal compound can be ^{99m}TcO₄⁻, ^{99m}TcO²⁺, ^{188m}ReO^{2 +}, ^{99m}TcO³⁺, ^{188m}ReO³⁺, or a compound including Fe, Ga, In, and the lanthanides. In addition, the metal compound can be initially chelated with a biocompatible ligand which is displaced by the recombinant pepcide chelate. The metal compound can be a charged or electroneutral complex having the formula (O−Me(V))₁L; wherein Me(V) is one of the gamma emitting isotopes of group VII transition metals; i is 1 to 4; and L can be a mono- or di-saccharide. L can also be saccharic acid, glucoheptonic acid, gluconic acid, glucuronic acid, glucooctanoic acid, sorbitol, glucosamine, or mannitol. In addition, L can be a mono- or polycarboxylic acids; e.g., tartaric, citric, or malonic acid.

In these methods, the host can be imaged, and the gene expression quantified, by an imaging technique such as magnetic resonance imaging, magnetic resonance spectroscopy, planar scintigraphy, single photon emission tomography, positron emission tomography, or X-ray computed tomography.

The host can be an animal, e.g., a mammal such as a human, non-human primate, horse, cow, pig, sheep, goat, dog, cat, mouse, rat, guinea, hamster, or ferret, a non-mammalian animal such as a chicken or frog, any other eukaryote, or a prokaryote.

In a further aspect, the invention features a system for measuring gene expression of a polypeptide or peptide in a host. The system includes a metal compound and an expression vector that includes a nucleic acid sequence encoding the polypeptide or peptide, and an imaging marker gene (IMG) encoding a recombinant peptide chelate (RPC) which chelates the metal compound.

As used herein, a “metal compound” is defined as any metal atom or ion, or charged or electroneutral compounds containing a metal atom or ion. The metal can be bonded by a covalent, ionic, or agostic interaction. An agostic interaction involves the coordination of a C—H bond with an unsaturated metal atom. Thus, metal compounds include, but are not limited to, oxocations, metal-ligand complexes, metalloproteins, organometallics, radioactive complexes, main group metal complexes, transition metal complexes, lanthanide complexes, actinide complexes, metal-RPC complexes, metal salts, clusters, and metalloproteins.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, technical manuals, and other references mentioned herein are incorporated by reference in their entirety. In case of conflict, the present application, including definitions, will control. In addition, the materials, methods, and examples are illustrative only and not intended to be limiting.

Advantages of the new methods, IMGs, and RPCs include: 1) the IMGs can be expressed in the same open reading frame with the gene of interest; 2) the RPCs allow selective labeling of recombinant peptides expressed in vivo with readily available metal compounds; 3) the resultant RPC-metal complexes are thermodynamically and kinetically stable; 4) the size of the RPC, and consequently the IMG, is quite small; and 5) the immunogenicity of the RPCs and RPC-metal complexes is low.

Other features and advantages of the invention will be apparent from the following detailed description, and from the claims.

### BRIEF DESCRIPTION OF THE DRAWINGS

FIG. 1 is a schematic illustration of a proposed model of the binding of an RPC (WGGC; SEQ ID NO: 1) to a metal compound.

FIG. 2 is a schematic illustration of a proposed model of the binding of an RPC (GGC-C; SEQ ID NO: 2) to a metal compound.

FIG. 3 is a reaction diagram for oxotechnetium reduction and chelation in vitro and re-chelation in vitro and in vivo.

FIGS. 4a and 4b are schematic maps of expression vectors.

FIG. 5 is a reaction diagram for the preparation of a plasmid.

FIG. 6 is a chromatograph obtained from high performance liquid chromatography (HPFC) of an RPC (WGGC) with ^{99m}Tc glucoheptonate labeling, imaged 30 minutes after introduction of the metal compound.

FIG. 7 is a chromatograph obtained from high performance liquid chromatography (HPLC) of an RPC (WGGC) with ^{99m}Tc glucoheptonate labeling, imaged 24 hours after introduction of the metal compound.

FIGS. 8 is a reproduction of an autoradiograph of a polyacrylamide gel.

FIGS. 9a and 9b are plots of fluorescence intensity of protein extracts (including labelled RPCs) normalized by protein content.

### DETAILED DESCRIPTION

The invention features a method for assessing gene expression (e.g., after gene therapy) by monitoring in vivo expression of an imaging marker gene (IMG) inserted into an expression vector (e.g., a plasmid) containing a desired gene, e.g., a therapeutic gene. The expression product of the IMG is a short peptide, termed “recombinant peptide chelate” (RPC), that binds a metal compound, such as a main group, lanthanide, or transition metal complex. Successful transfection of a cell with the expression vector results in expression of both a desired protein, such as a therapeutic protein, and the RPC, either as a therapeutic fusion protein containing the RPC, e.g., at one or the other terminal end, or as a separate therapeutic protein and peptide.

The RPC is expressed on the cell surface or in the extracellular space when the therapeutic gene is expressed. Thus, by administering a dose of the metal compound to a patient and imaging to detect metal-RPC complexes which form, it is possible to deduce whether or not the therapeutic gene was expressed. An advantage of the method is that it provides qualitative and semiquantitative anatomical gene expression data without the need for invasive procedures, such as tissue sampling.

Recombinant Peptide Chelates (RPCs)

RPCs can include any combination of natural or artificial amino acids, bound in a linear or branched peptide configuration, which folds to generate a metal binding site capable of association with a metal compound (as defined herein) as a result of favorable redistribution of electron density. In some cases, the peptides can require post-translational modification within the host cell to generate this site. Examples of such modification include O- or N-glycosylation, palmitoylation, myristoylation, farnesylation, or phosphatidylinositol-glycan (PI-G) linkage. Generally, the actual binding site includes only about three or four amino acids, although longer peptides can be used to lock those three or four amino acids into the correct conformation for binding one or multiple metal compounds.

RPCs can have the generic structure shown in formula I:

(XGₐC)_{b}Z_{c}CG_{d}X (I)

where X can be any amino acid or amino acid sequence, for example up to 10, 20, or even up to 1000 amino acids or more, Gₐ is one, two, three, or four glycine residues (a is 1 to 4) , C is cysteine, Z_{c} can be absent or any amino acid or amino acid sequence of up to four amino acids (e.g., valine, proline, or glycine, or combinations thereof) (c is 0 to 4) , G_{d} is one, two, three, or four glycine residues (d is 1 to 4), and b is 1 to 2. The generic structure of formula I can occur just once, or can be repeated one or more times in the same molecule, linked, e.g., by peptide bonds.

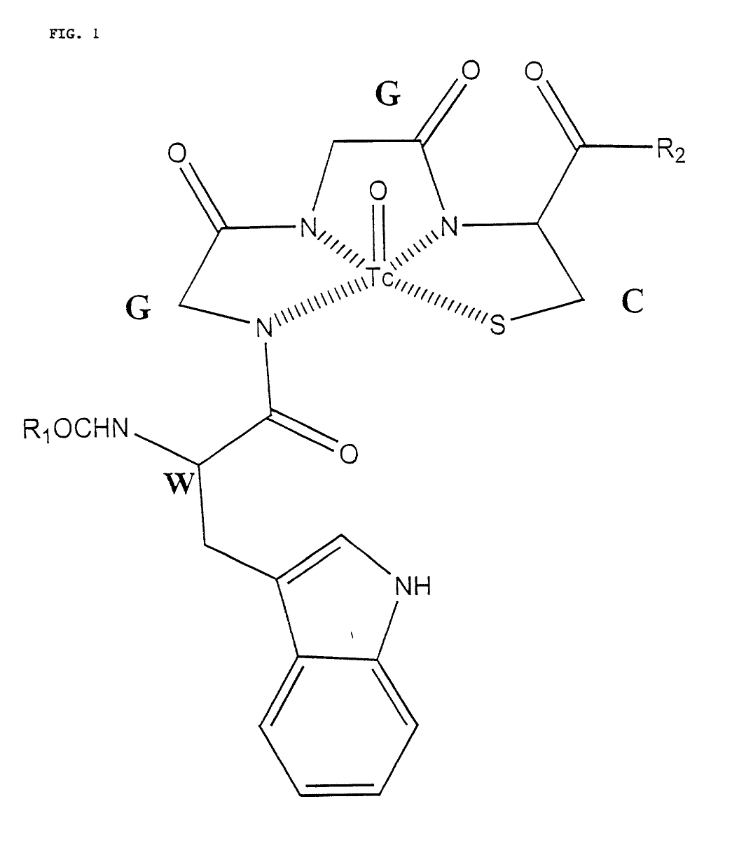

Each XGₐC motif can bind to a metal compound (e.g., Tc, Re, Fe, Ga, In, or lanthanide compounds). For example, the RPC amino acid sequence JWGGCJ (SEQ ID NO: 3); where J is either aspartic acid or glutamic acid, and W is tryptophan; binds ^{99m}TcO²⁺. FIG. 1 is an illustration of a proposed model of RPC core amino acids (WGGC; SEQ ID NO: 1) of this binding Interaction. The interaction of the RPC with the pertechnetate ion results in strong chelation of the metal oxocomplex by the polypeptide.

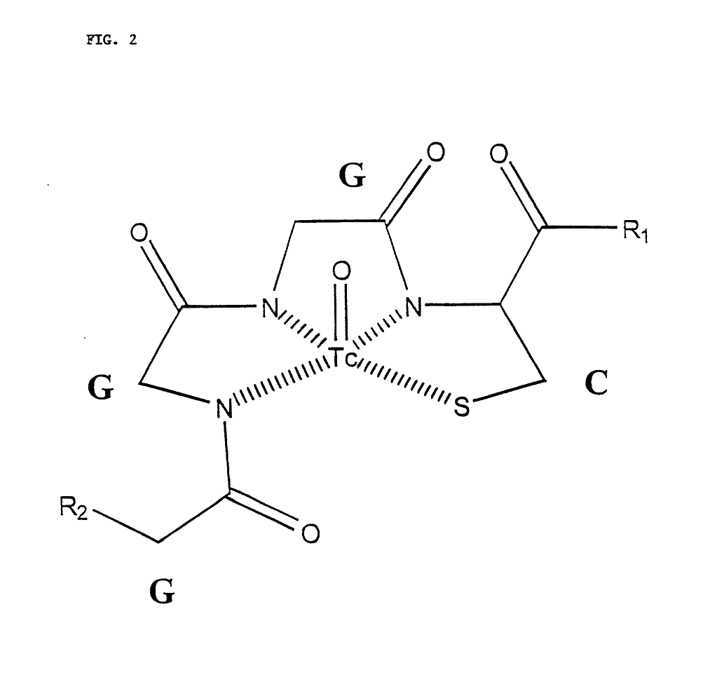

FIG. 2 illustrates the binding interaction of a proposed model of another RPC core, GGGC (SEQ ID NO: 2), which shows coordination of Tc(V)O by the GGGC motif. The high affinity of oxotechnetate interaction with GGGC leads to re-chelation of oxotechnetate from a complex with other ligands, such as glucoheptanoic or glucaric acids, allowing visualization of the presence of these motifs in recombinant peptides either in situ or in vivo. The latter is more attractive for the purpose of non-invasive detection of spatial distribution and levels of foreign gene expression in vivo, e.g., to monitor gene expression during gene therapy.

Imaging Marker Genes (IMGs) and Therapeutic Genes

In general, imaging marker genes (IMGs) include a nucleic acid sequence encoding an RPC (metal-binding amino acid sequence), and a promoter sequence (if there is not already a promoter sequence in the expression vector). These short IMGs can be easily synthesized using standard techniques and equipment. See, e.g., Ausubel et al., 1995, Current Protocols in Molecular Biology (John Wiley & Sons, Inc., N.Y., 1995). The IMGs can additionally include other nucleic acid sequences, such as restriction sites or sequences encoding cell surface trafficking peptides, membrane anchoring domains, or secretion signals. Suitable promoters include the adenovirus major late promoter, early and late promoters of SV40, CMV promoter, TH promoter, RSV promoter, or B19p6 promoter (Shad et al., J. Virol., 58:921, 1986). The promoter may additionally include enhancers or other regulatory elements.

The so-called “desired” genes, e.g., therapeutic genes, used in the new methods, are any genes that can be expressed in mammalian or other cells. For a review of gene therapy and various promoters used in gene therapy, see, e.g., Walther et al., J. Mol. Med., 74:379-392 (1996). For example, these therapeutic genes can be genes encoding blood coagulation factors such as Factor VIIIc (e.g., as described in Toole et al., Nature, 312:342, 1984; Wood et al., Nature, 312:330, 1984), anti-thrombin III, or Factor IX (Kurachi et al., PNAS, 79:6461, 1982), growth factors such as transforming growth factor alpha (TGF-α), hormones, interleukins, interferons, tyrosine kinase, adenosine deaminase (Valerio et al. Gene, 31:147, 1984), α-1 antitrypsin (Ciliberto et al. Cell, 41:531, 1985), or the cystic fibrosis transmembrane conductance regulator (Riordan et al., Science, 245:1066, 1989).

These therapeutic genes, i.e., the nucleic acid sequence that encodes the therapeutic protein or peptide, can be inserted into the expression vector so that they are under the control of the same promoter as the sequence encoding the RPC (i.e., to yield a fusion protein or peptide), but can alternatively be placed under the control of a second copy of the same promoter, or under the control of a separate promoter. The latter alternatives afford separate proteins or peptides and can therefore be useful if the RPC interferes with the function of the therapeutic protein or peptide (e.g., in protein folding).

Especially useful for preparation of fusion proteins are expression vectors that contain a nucleotide sequence that can be digested at a restriction site and ligated in the same open reading frame with a protein of interest (therapeutic gene product), for example:

| | |
|---|---|
| G \| GG ATC [GAA GGA GGA GGA TGT]ₙ [GAA GGA GGA TGT]ₘ | (SEQ ID NO: 4), |

which encodes:

| | |
|---|---|
| BamHI rs [Glu Gly Gly Gly Cys]ₙ [Glu Gly Gly Cys]ₘ | (SEQ ID NO: 5) |

The vertical line in SEQ ID NO: 4 represents a BamHI cleavage site. The oligonucleotide can include repeating elements n or m, or both in the same sequence, where n and m can be one or more. The expression of the fusion protein in mammalian cells will result in a product that can bind metal compounds with high affinity.

Preparation of Recombinant Peptide Chelates and Insertion of Imaging Marker Genes into Expression Vectors

The production of recombinant polypeptides bearing metal binding peptide sequences can be achieved by standard methods of genetic manipulation. For example, new IMGs can be introduced into plasmid vectors, episomal vectors, viral amplicons, or numerous other expression vectors (see, for example, Balbas et al., Methods Enzymol., 18:14, 1990, or Miller, L. K., Curr. Opin. Genet. Dev. , 3:97, 1993). IMGs can also be introduced by insertion or deletion of specific genomic elements, such as DNA sequences, accomplished by induction of splicing or self-splicing of the host genome. Alternatively, synthetic or natural mRNA encoding RPCs can be introduced directly into the expression vector.

In any of the above examples, the polymerase chain reaction (PCR) can be used to inexpensively “mass-produce” both the therapeutic gene and the IMGs to be used for targeted gene expression (e.g., for gene therapy). See, e.g., Ausubel et al. (supra).

Preferably, the IMG is inserted at a specific site in the same open reading frame (ORF) of an expression vector as the sequence encoding either a membrane anchoring domain (e.g., glycosylphosphatidylinositol-linked protein Thyl thymocyte marker, or phosphatidylinositol-glycosylation signal from alkaline phosphatase) (Gerber et al., J. Biological Chem. 267(17):12168-73, 1992), or a secretion signal derived from proteins such as prolactin, growth hormone, the insulin-like factors, or the interleukins. These anchoring domains and secretion signal sequences can be obtained and inserted into expression vectors using standard techniques.

Provided that the IMG encoding the RPC is positioned under the control of a strong promoter (e.g., SV40 promoter or CMV promoter) in the expression vector, the RPC expression product will be produced by a transfected cell. As mentioned above, the same promoter can also control expression of another gene of interest (e.g., a therapeutic gene), in which case the expression product will also include the therapeutic protein. Alternatively, the synthetic gene encoding the RPC can be inserted into an expression vector along with the therapeutic gene under the control of two separate promoter elements. In still another alternative, the RPC can be supplied in an entirely separate expression vector introduced to the host concurrently with the expression vector containing the therapeutic gene.

Introduction of Expression Vectors into Hosts

For in vivo gene transfer and manipulation, the expression vectors described above can be introduced into a host (e.g., an animal, such as a human or domesticated animal such as a horse, dog, cow, pig, or chicken) by numerous known methods. Such methods include the use of replication deficient viral particles (see e.g., Sambrook et al., Molecular Cloning, Vol. 3, Cold Spring Harbor Press, N.Y., 1989); non-viral containers of genetic material, such as liposomes or liposome mimetics, which can be conjugated with targeting ligands (Fraefel et al., J. Virology, 70:7190, 1996); polymeric or copolymeric electrostatic complexes; delivery by direct injection of genetic material into the tissue of the host (Manthorpe et al. Hum. Gene Therapy, 4:419, 1993); and in vivo electroporation of cellular membranes.

For example, a sterile solution containing an expression vector encoding a therapeutic protein fused with an RPC can be mixed with a sterile solution containing balanced or hyperosmotic salts; saline; and biocompatible, endotoxin-free carrier components, including lipids, proteins, linear polymers, graft co-polymers, polymer-coated nanoparticles, or liposomes, or a combination these components. The final concentration of the expression vector in the total solution can be about 0.05-10% by weight. The composition can be incubated for about 5-30 minutes to allow time for the formation of a complex between the DNA and the carrier. The composition can then be introduced aseptically into the host via intraarterial, intravenous, subcutaneous, or direct (i.e., into a tissue) injection.

Alternatively, cells can be genetically modified ex vivo and then introduced into a host by, for example, injection or implantation. When cells are to be genetically modified for the purposes of ex vivo manipulation or gene transfer, the vectors described above can be introduced into cells (e.g., human primary or secondary cells, such as fibroblasts, epithelial cells including mammary and intestinal epithelial cells, endothelial cells, formed elements of the blood including lymphocytes and bone marrow cells, glial cells, hepatocytes, keratinocytes, muscle cells, neural cells, or the precursors of these or any other malignant cell types; non-human animal cells; plant cells; other eukaryotic cells; or prokaryotic cells) by standard methods of transfection including, but not limited to, liposome-, polybrene-, or DEAE dextran-mediated transfection, electroporation, calcium phosphate precipitation, microinjection, or velocity driven microprojectiles (“biolistics”). See, for example, Sambrook et al., (supra).

Alternatively, one could use a system that delivers DNA by viral vector. Viruses known to be useful for gene transfer include adenoviruses, adeno associated virus, herpes virus, mumps virus, parvovirus, poliovirus, retroviruses, Sindbis virus, and vaccinia virus such as canary pox virus. See, for example, Cohen et al., PNAS, 90:7376, 1993; Cunningham et al., Virology, 197:116, 1993; or Halbert et al., J. Virol., 69:1473, 1995.

For model studies of tumors, one can also use immortalized human cells. Examples of immortalized human cell lines useful in the present methods include, but are not limited to, Bowes Melanoma cells (ATCC Accession No. CRL 9607), Daudi cells (ATCC Accession No. CCL 213), HeLa cells and derivatives of HeLa cells (ATCC Accession Nos. CCL 2, CCL 2.1, and CCL 2.2), HL-60 cells (ATCC Accession No. CCL 240), HT1080 cells (ATCC Accession No. CCL 121) , Jurkat cells (ATCC Accession No. TIB 152), KB carcinoma cells (ATCC Accession No. CCL 17), K-562 leukemia cells (ATCC Accession No. CCL 243), MCF-7 breast cancer cells (ATCC Accession No. BTH 22), MOLT-4 cells (ATCC Accession No. 1582), Namalwa cells (ATCC Accession No. CRL 1432), Raji cells (ATCC Accession No. CCL 86), RPMI 8226 cells (ATCC Accession No. CCL 155), U-937 cells (ATCC Accession No. CRL 1593), WI-38VA13 subline 2R4 cells (ATCC Accession No. CLL 75.1), and 2780AD ovarian carcinoma cells (Van der Blick et al., Cancer Res., 48:5927-5932, 1988), as well as heterohybridoma cells produced by fusion of human cells and cells of another species. Secondary human fibroblast strains, such as WI-38 (ATCC Accession No. CCL 75) and MRC-5 (ATCC Accession No. CCL 171), can also be used.

Administration of Metal Compounds Into Hosts

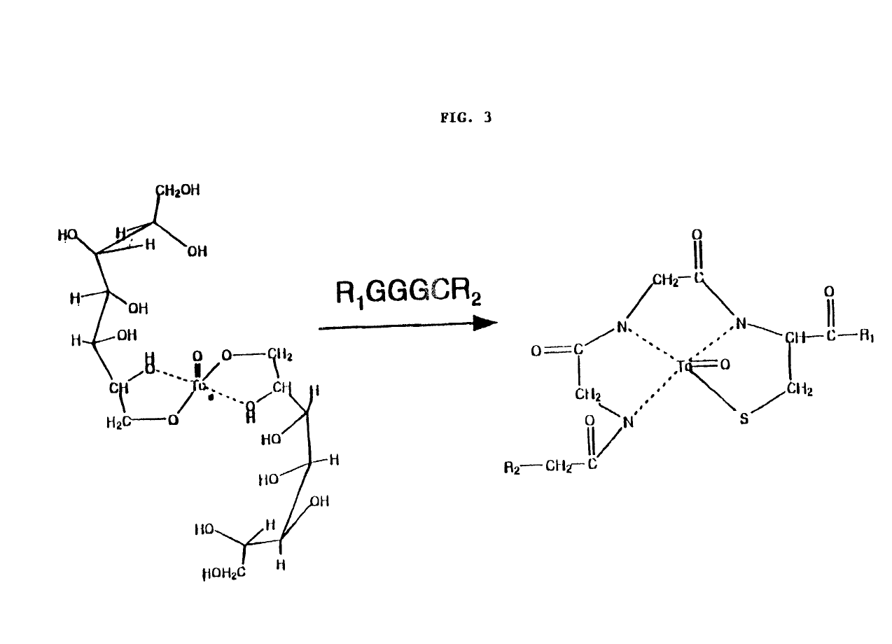

Metal compounds can be introduced into a host by local or systemic means, e.g., intramuscular, intravenous, or intra-arterial injection, often with the metal bound to low molecular weight stabilizing ligands, such as mono- or di-saccharides. These ligands are specifically chosen with two criteria in mind: 1) the ligands must reduce the probability that the metal compounds will become associated with non-specific sites (e.g., in the plasma protein components or non-affected cells), and 2) the ligands must have low enough metal-binding affinity relative to the RPCs such that they are easily displaced by the latter. FIG. 3 is an illustration of one such re-chelation reaction, wherein glucoheptonate is displaced by a GlyGlyCys binding domain.

Because the metal-ligand complexes have low molecular weight, they are capable of both permeating the interstitia and also crossing vascular barriers by diffusion or convection. Within the interstitium, the presence of transfected cells results in a high local concentration of RPC metal-binding sites. The RPCs then displace the low-affinity stabilizing ligands and re-chelate the metals. The displaced ligands leave the host primarily via the kidneys. Excess metal compound (i.e., that which does not become complexed with RPCs) is also cleared by the kidneys in most cases.

Due to the short biological half-life of typical low molecular weight complexes of transitional metals and lack of specific site of accumulation (excluding kidneys), the optimal waiting time between the injection and imaging procedures is typically about 1 to 3 hours. This is also true for compounds prepared from most medically useful radioactive metals, given their relatively short half-lives for decay (e.g., t_{1/2}=6 hours for ^{99m}Tc)

Assaying for Gene Expression

The expression of the therapeutic gene supplied for targeted expression in a host in conjunction with the expression of the RPC can be detected by in vitro methods or in vivo methods. One in vitro method includes analyzing a sample of biological material with a metal binding assay, in which the sample is mixed with a suitable metal compound to form complexes, and then isolating the resulting complexes. The isolated complexes can be analyzed by standard methods, such as the detection of the radioactivity associated with the sample (i.e., if a radioactive metal was employed). Alternatively, the complexes can be analyzed by detecting the change in water proton relaxation rates in an NMR experiment.

Alternatively, the detection is accomplished in vivo within the host, after the local or systemic introduction of a solution containing the metal compound with or without a stabilizing ligand, as described herein. The detection and quantitation of radioactivity (e.g., with a gamma camera) or a change in water proton relaxation rates (e.g., with magnetic resonance imaging) indicates the localization of the expressed gene product in the body. The in vivo method is advantageous in that it is non-invasive and it provides semiquantitative, anatomical data. See, for example, Henkin et al., “Nuclear Medicine,” Mosby, St. Louis (1996); or Edelman et al., “Clinical Magnetic Resonance Imaging,” Saunders, Phila. (1996). Suitable methods for imaging the host and quantitating gene expression include, but are not limited to, magnetic resonance imaging (MRI), magnetic resonance spectroscopy, planar scintigraphy, single photon emission tomography, positron emission tomography (PET), and X-ray computed tomography (CT).

The following are examples of the use of the new methods and compositions.

### EXAMPLE 1

Preparation of an IMG Encoding a Specific Metal-Binding Peptide Sequence

The green fluorescent protein (GFP) encoding sequence (Prasher et al., Gene, 111:229-233, 1992; Chalfie et al., Science, 263:802-805, 1994) was amplified by PCR, using 1) a forward primer bearing the EcoRI restriction site immediately upstream of a Kozak sequence and Met initiation codon (5′-GGAAGCTTGAATTCTGCCGCCACCATG-3′) (SEQ ID NO: 6); and 2) reverse primers encoding P1 (LEGGGCEGGC) (SEQ ID NO: 7) and P2 (LGGGGCGGGCG) (SEQ ID NO: 8) metal-binding RPC sequences.

### EXAMPLE 2

Human Placental GPI Signal Amplification and Cloning

The hydrophobic fragment of human placental alkaline phosphatase (PLAP) protein includes 29 C-terminal amino acids, with aspartic acid 484 (Asp-484) serving as a glycophosphatidylinositol (GPI) addition site. The full-length HindIII-XbaI 5.0 kilobase (kb) fragment of the PLAP gene (i.e., the internal fragment of the PLAP gene remaining after digestion with the restriction enzymes HindIII and XbaI) was isolated from the pRSVPAP plasmid (ATCC Accession No. 77129-77131, Rockville, Md.). PCR was also used to obtain this 108 nucleotide fragment.

To create a new BamHI restriction site for further linkage with a tyrosinase fragment, the following mutagenic oligonucleotide was chosen: 5′-GCCTGCGACCTGGGGATCCCCGCCGGCA CC-3′ (SEQ ID NO: 9), where the induced mutations are shown in bold-face type. Another synthetic oligonucleotide from the PLAP 3′-terminal end has the sequence: 5′-CTCAGGGAGCAGT GGCGTCTCCAGCAGCAG-3′ (SEQ ID NO: 10). This small PCR product therefore included a DNA sequence corresponding to a 35 residue peptide encompassing the 29 amino acid PLAP hydrophobic region, the Asp-484 GPI addition site, the five amino acids upstream of Asp-484, and a stop codon site.

The PLAP PCR fragment was digested with BamHI and cloned into the pBluescript (pBS) KS vector. The vector plasmid was digested with EcoRI, the sticky ends were blunted by Klenow digestion, and the resulting polynucleotide was digested with BamHI. Clones were screened using PCR and the primary structure was verified by sequencing.

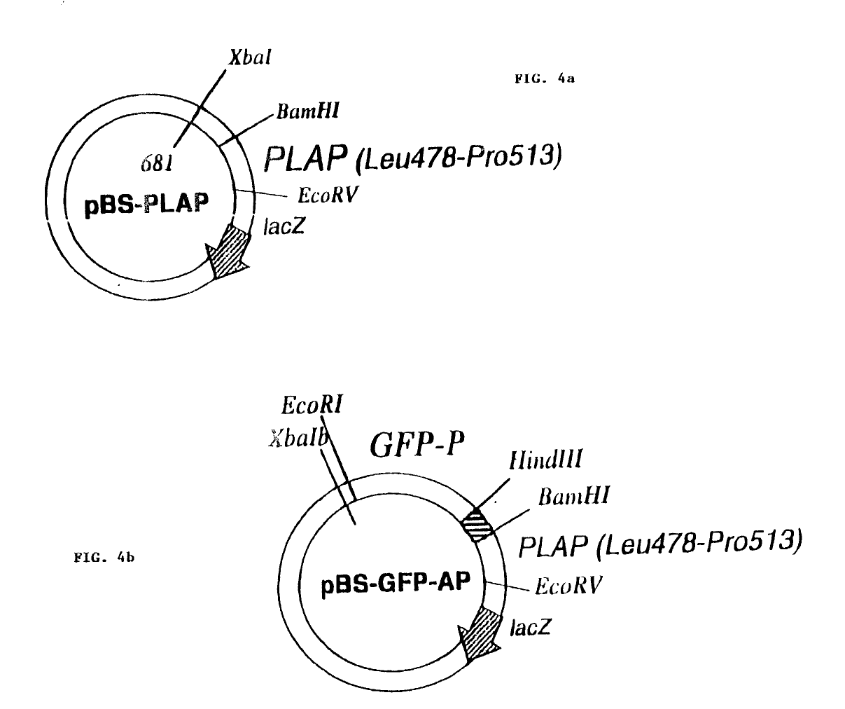

The pBS-PLAP.1 clone, as it was termed, was selected for further ligation with GFP amplified fragments. The plasmid (pBS-PLAP), mapped in FIG. 4a, was digested with XbaI restrictase, blunted by Klenow digestion, then digested with BamHI, and ligated with a BamHI-digested fragment obtained from the PCR-amplified GFP-P gene. Clones, termed pBS-GFP-AP, were screened by PCR. Primary structure was proved by sequencing.

### EXAMPLE 3

Cloning of GFP-P-AP Into the Eukaryotic Expression Vector pZeoSV

The PBS-GFP-AP clone, mapped in FIG. 4b, was selected as described above for recloning into the pZeoSV expression vector (INVITROgen) under the control of the SV40 promoter. pZeoSV plasmid contains a Zeocin (bleomycin) resistance gene under the control of the CMV promoter (2184-2802), which serves as a selective marker.

The XhoII 1.4 kb CMV promoter fragment was isolated from the pZeoSV vector and recloned downstream from the SV40 promoter into the same plasmid vector. PCR amplification was carried out with the aid of primers which allow the creation of HindIII and EcoRI restriction sites:

| 5′ -GATCTAAGCCCTTCGTTACATAACTTACG-3′ | (SEQ ID NO: |
|---|---|
| | 11) and |
| 5′ -CACGTGCTGGAATTCCGTTCCAATGCACCG-3′ | (SEQ ID NO: |
| | 12), |

respectively, where the induced mutations are shown in bold-face type. The CMV promoter fragment was then digested with HindIII and EcoRI and recloned into the pZeo-GFP-P-AP plasmid which had previously been treated with the same restriction enzymes. The clones were screened by HindIII-EcoRI restriction digestion, followed by PCR. The selected pZeo-GFP-P-AP clone contained the whole DNA construct sequence under control of the CMV/SV40 double promoter.

### EXAMPLE 4

Cloning of GFP-P-AP Into the pCDNA3 Vector

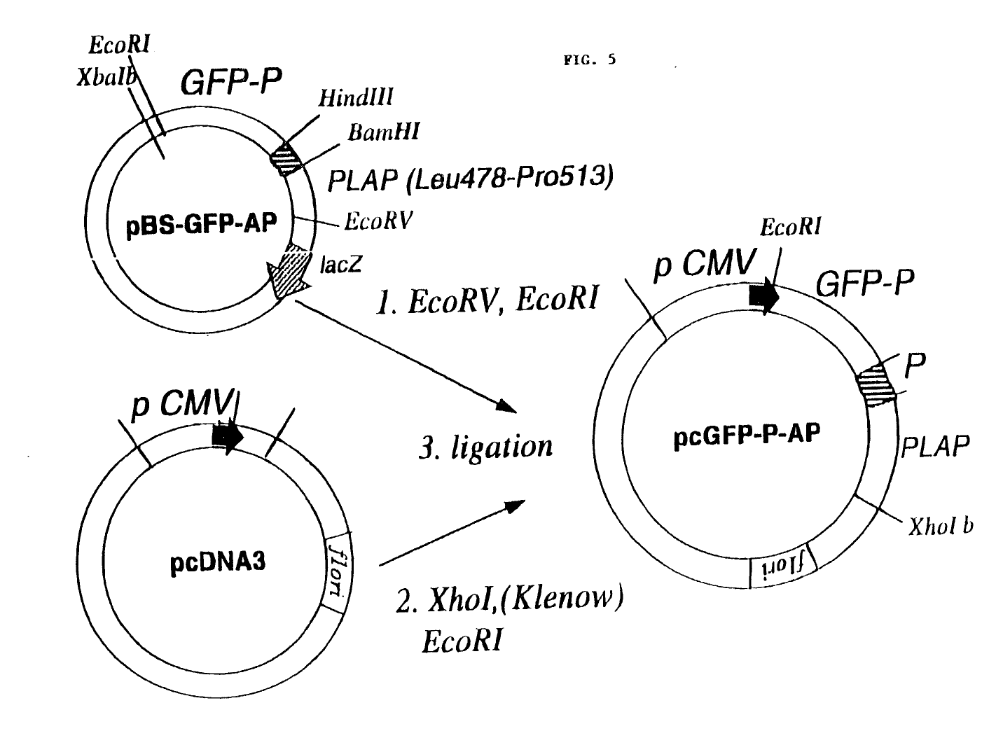

The pBS-GFP-AP plasmid was digested with EcoRV and EcoRI and the GFP-P-AP insert was isolated by preparative gel electrophoresis. The eukaryotic expression vector pCDNA3 was digested with XhoI restrictase, blunted by Klenow digestion, and digested with EcoRI. The GFP-P-AP fragment was ligated with digested pCDNA3 vector in the presence of T4 DNA ligase to give a vector termed pcGFP-P-AP. A diagram which illustrates the preparation of this vector is provided in FIG. 5.

### EXAMPLE 5

Transfection of COS-1 Cells in Vitro With Experimental and Control Expression Vectors

COS-1 cells were transfected at a concentration of 7-10×10⁴ cells/well by either 5 μg pcGFP-P-AP experimental vector or 5 μg pCDNA-GFP control vector. The vectors were included in a calcium phosphate precipitate or mixed with DEAE dextran before adding to cells according to the procedure of Sambrook et al. (supra) . In both experimental (pcGFP-P-AP-transfected) and control (pCDNA-GFP-transfected) cells the expression of fluorescent product was detected 16 hours after transfection. In the pcGFP-P-AP-transfected cells, membrane-bound fluorescence of the GFP marker gene was found to be evident in the regions of membrane ruffling.

### EXAMPLE 6

Transfer of the Expression Vector Into Mammalian Cells in Vivo Without Viral Particles

A sterile solution containing an expression vector encoding GFP fused with P-AP is mixed with a sterile solution containing balanced salts, saline, and biocompatible, endotoxin-free lipidic carrier components. The final concentration of the expression vector in the total solution is about 1% by weight. The composition is incubated for 30 minutes to allow for the formation of a complex between the DNA and carrier. The composition is then introduced aseptically via subcutaneous injection.

### EXAMPLE 7

Transfer of the Expression Vector Into Mammalian Cells in Vivo with Viral Particles

DNA encoding pcGFP-P-AP is inserted into the viral amplicon vector pHSVPrPUC containing viral noncoding sequences (i.e., origin of replication and specific packaging signal sequences). DNA encoding interleukin 2 (IL-2) is also inserted into the plasmid in the same ORF as pcGFP-P-AP. Q-2 packaging cells are transfected with the viral amplicon and then infected with a replication-deficient helper virus or transfected with infectious viral DNA. Viral amplicon vectors produced by the transfected cell line are collected; passaged; assayed for the amplicon, undesirable helper virus, and wild type virus contents; and used for gene delivery in vivo via intraarterial, intravenous, subcutaneous, or direct (i.e., into a tissue) injection.

### EXAMPLE 8

In Vivo Detection of Gene Expression Products Including RPCs

After the 1 to 8 day waiting period required for gene expression, a subject previously infected with a virus of Example 7 is injected intravenously with a sterile solution of an ¹¹¹In oxocomplex with a disaccharide ligand. After a period of 20 to 60 minutes, whole body or collimated target organ images are collected using a gamma camera and standard techniques. The images of the RPC-¹¹¹In complexes indicate where the IL-2 has been expressed. The intensity of the images correlates to the amount of the IL-2 expressed in vivo.

### EXAMPLE 9

In Vivo Detection of RPCs in Muscle Tissue

RPCs having the sequences GGGCGGGCGGGC (SEQ ID NO: 13) and GGGCGGGC (SEQ ID NO: 14) were dissolved in sterile saline to give a concentration of 1 μM thiol (i.e., the —SH function groups of cysteine residues). 100 μl of this solution was injected into the rectus femoris muscles of anesthetized rats. Ten minutes after injection, 0.5 mCi of ^{99m}Tc gluceptate-[bis(oxotechnetate(v)glucoheptonate] anion was injected via intravenous catheter. The animals were imaged at 10 minutes and at 1 hour after the injection of the isotope. It was suspected that Tc glucoheptonate can permeate into the interstitium and be taken up non-specifically (e.g., by pinocytosis) by resident cells, thus creating background activity.

Nonetheless, the radioactivity that had accumulated in the injected muscle 1 hour after injection with the RPC containing three consecutive GGGC repeats (SEQ ID NO: 13) or two consecutive GGGC repeats (SEQ ID NO: 14) were 3- or 2-fold higher, respectively, than the radioactivity that had accumulated in the contralateral (control) muscle at that time. No differences in radioactivity accumulated in injected vs. control muscles were detected in animals injected with saline, or an RPC pretreated with N-ethylmaleimide to block oxotechnetate-binding sites.

### EXAMPLE 10

Stability of Metal-RPC Complexes

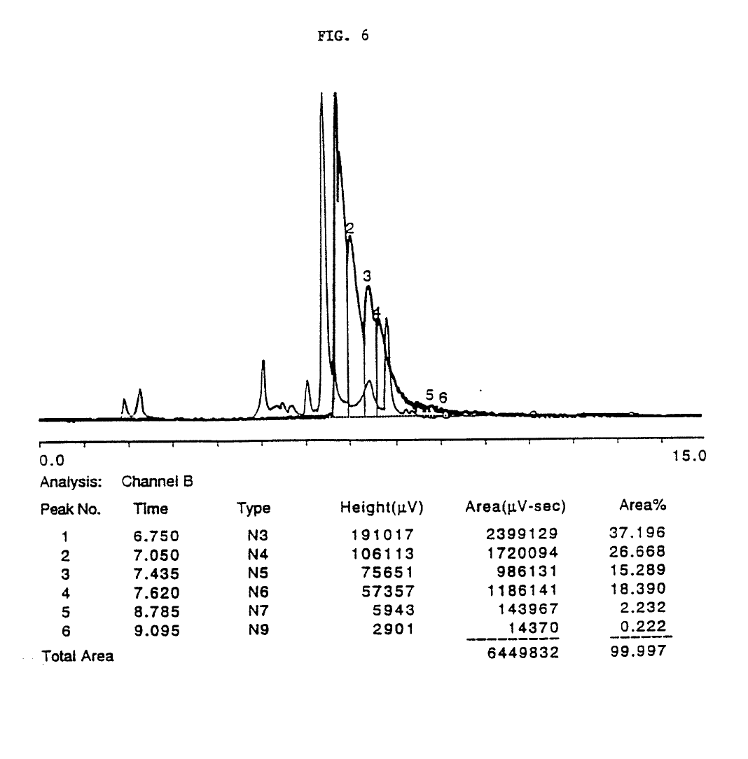

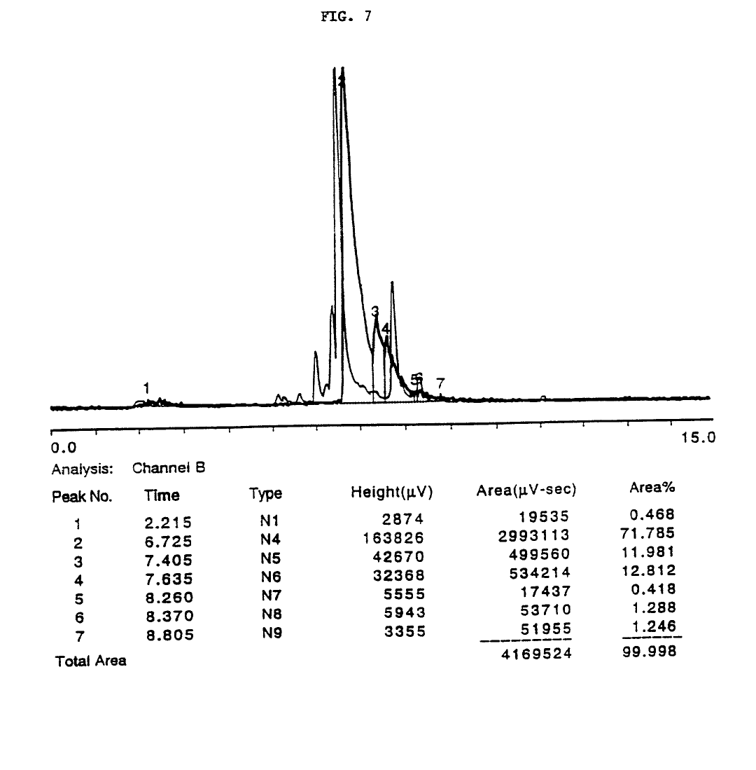

The tetrapeptide WGGC (SEQ ID NO: 1) was incubated with ^{99m}Tc-gluccheptonate. The incubation unexpectedly resulted in very high (99%) labeling of the peptide within 30 minutes (see, FIG. 6). The label was ascertained to be stably associated with the peptide for at least 24 hours as determined by reverse-phase high performance liquid chromatography (HPLC) (see, FIG. 7).

FIGS. 6 and 7 are the HPLC traces corresponding to the binding 30 minutes and 24 hours post-injection, respectively. In both traces, the bold line represents radioactivity and the thin line represents absorbance. The naked ^{99m}Tc-glucoheptonate has a retention time of 2 minutes, while the RPC-Tc complex has a retention time of 5 to 9 minutes. FIG. 6 shows that at 30 minutes post-injection, no radiation is observed above the baseline at 2 minutes. FIG. 7 shows that at 24 hours post-injection, only 0.468% of the total integral (see, peak 1 in the integral data) occurs at 2 minutes.

### EXAMPLE 11

Preparation of a Membrane-Anchored Peptidase-GFP-RPC Construct

A new IMG, encoding GFPP3, was prepared using the GFP encoding sequence and PCR as described in Example 1, but using reverse primer P3. The coding DNA sequence of GFPP3 was amplified with PCR using a sense primer containing an internal EcoRI site: 5′-ggaagcttgaattcaccatggtgagcaaggg-3′ (SEQ ID NO: 15) and a reversed primer (antisense) encoding a C-terminal RPC (P3): LeuGluGlyGlyCysProCysGlyGlyGlyIle (SEQ ID NO: 23) and bearing a terminal BamHI restriction site: 5′-caggatccctcctccacatggacatcctcctccaagcttgtacagctcgtccatgccg-3′ (SEQ ID NO: 24). The PCR fragment was subcloned into the BSKS vector using EcoRI and BamHI sites.

A 300 bp fragment of rabbit neutral endopeptidase-24.11 containing a NH2-terminal transmembrane domain (23 aminoacids) and a signal peptide (27 aminoacids) was isolated from pSVENK19 by digestion with MspI and PvuII and subcloned into BSKS-GFPP3 digested with AccI and EcoRI (blunt).

Selected clones were treated by XhoI(blunt)-XbaI and cloned into HindIII(blunt)-XbaI sites of expression vector pCDNA3. The new construct provides an N-terminal transmembrane domain to anchor the RPC to a cell membrane.

### EXAMPLE 12

Expression of an RPC in Prokaryotic Cells

A Bluescript GFP-bearing plasmid (Prasher et al., Gene, 111:229-233, 1992; Chalfie et al., Science, 263:802-805, 1994) was obtained and a C-terminal fusion was made by PCR, using 1) Pwo I polymerase; 2) a sense primer containing an EcoRI restriction site (5′-ggaagcttgaattcaccatggtgagcaag gg-3′) (SEQ ID NO: 15); and 3) an antisense primer with BamHI and Hind III restriction sites: (5′-caggatcccacatcct cctccacatcctcctcctccaagcttgtacagctcgtccatgcc-3′) (SEQ ID NO: 16), the latter encoding a peptide with two GlyGlyCys motifs (LGGGGCGGGCCI) (SEQ ID NO: 17), termed “hydrophobic GFP-P2.” Alternatively, a primer encoding a negatively charged peptide (LEGGGCEGGC) (SEQ ID NO: 18) was used: (5′-caggatcctaacatcctccttcacatcctccaagcttgtacagctcgtccatg cc-3′) (SEQ ID NO: 19), termed “hydrophilic GFP-P1.”

The PCR products were purified, digested with the restriction enzymes EcoRI and BamHI, and inserted into a BSKS(+) vector (Stratagene). The amino-terminal sequence of β-galactosidase upstream of the GFP AUG codon was partially excised with the restriction enzymes Acc65I and EcoRI. The resulting sticky ends were blunted by Klenow digestion and ligated in the open reading frame of the lac Z gene.

A control construct was prepared by excising the oxotechnetate-binding C-terminal peptide using HindIII and BamHI with subsequent ligation of sticky ends. Competent E. coli DH5αcells were transformed with constructs bearing inserts encoding three GFP variants, and corresponding fluorescent clones were obtained by selecting colonies on ampicillin-treated agar. DNA minipreps from the isolations were analyzed for primary structure of 3′ and 5′ termini of GFP fusions, using sequencing from T3 and T7 primers (Sambrook et al. Molecular Cloning, “Ch. 13 DNA Sequencing,” 1989).

The cells were grown in Luria Broth (LB) overnight; washed with a 0.1 M Tris, 0.1 M NaCl solution at pH 7.5; and subjected to two different lysis procedures. In the first procedure, 0.25 g of cells were lysed in 500 μl of 0.05 M Tris, 2% SKS, 20 mM DTT, 10 μM CaCl2, and 20 μg/ml DNAse I at pH 8 for 1 hour, followed by addition of 100 μl of 0.5 MEDTA. In the second procedure, the cells were lysed with 1 mg/ml lysozyme in 0.05 M Tris 50 mM octyl-thioglucopyranoside, 1 mM PMSF, and 20 mM DTT for 1 hour at 4° C. Cells were then disintegrated by ultrasonication (on ice or 30 seconds) and the lysates were sedimented at 15,000×g for 20 minutes.

Protein content was analyzed using a BCA kit (Pierce) according to the manufacturer's directions. Lysates prepared in the presence of SDS were used for PAGE analysis and binding studies. The PAGE was carried out on 0.1% SDS, 12% polyacrylamide gels, with 10 mM thioglycolic acid in the running buffer. In some experiments lysates were also treated with a 10-fold excess of N-ethylmaleimide over DTT to block oxotechnetate-binding sites. The gels were fixed in a methanol-acetic acid mixture, then washed with 0.1 M Tris, 25% ethanol at pH 8.8.

10-20 mCi/0.5 ml of ^{99m}Tc-pertechnetate (Syncor) was added to a Glucoscan kit (DuPont Radiopharmaceuticals), diluted to 10 μCi/ml with 0.1 M NaCl, and agitated with the gel for 1 hour. Gels were washed in a solution containing 10 mM morpholinoethane sulfonic acid (Mes) and 0.15M NaCl at pH 6.5, dried, and subjected to autoradiography using Cronex film (5 minutes at room temperature with intensifying screen). Developed films and PAGE gels stained with colloidal Coomassie dye (ICN) were subjected to densitometry using BioMAX software (Kodak) for data analysis. Octylthioglucoside lysates were used for HPLC analysis using a Hydropore AX anion-exchange column (Rainin Inst. Co.) eluted with a gradient of 10 mM-2 M ammonium acetate, 10 mM DTT at pH 8. Fractions were collected and fluorescence was measured at λₑₓ 475/λₑₘ 508 in 0.5% SDS, 10 mM DTT at pH 8.

Three major types of GFP variants were cloned into EcoRI-BamHI sites of BSKS vectors. First, “hydrophobic GFP-P” was designed to bear a tandem repeat of relatively hydrophobic metal-binding regions (GGGC). “Hydrophilic GFP-P” was designed to include two residues of glutamic acid in an attempt to create an electrostatic repulsion between the fusion protein and the negatively charged glucathione disulfide which participates in thiol-disulfide exchange reactions (Gilbert, H. F. Methods Enzymol. 251, 8-28, 1995).

Cloning of GFP-P into the BSKS vector with subsequent sequencing allowed determination of the primary amino acid sequence of the fusion protein. A translation of the DNA sequence in the ORF of β-galactosidase for clone 5 is represented by: TMITPSAQLTLTKGNKRWVQPTMet{GFP}LGGGGCGG GCGI (N-terminal amino acids derived from the β-galactosidase sequence are in italics, plus Met (SEQ ID NO: 20); metal-binding repeats are in bold-face type (SEQ ID NO: 17)).

A spontaneous deletion of the 5′-end corresponding to eight N-terminal amino acids was revealed in one of the isolates (clone 6). In this clone, the AUG codon of GFP-P fusion protein is immediately adjacent to the BSKS(+) T3 promoter region. The corresponding translation of the fusion protein sequence is represented by: TMITPSAQL TLTKGMet{GFP}LGGGGCGGGCGI (SEQ ID NO: 21-{GFP}-SEQ ID NO: 17). These two variants of “hydrophobic” fusion proteins were studied separately for their ability to form complexes with oxotechnetate.

Sequencing of GFP variants fused to the “hydrophilic,” negatively charged C-terminal LEGGGCEGGC (SEQ ID NO: 22) peptide (Clone 3) revealed the same N-terminal amino acid sequence as in Clone 5. Additionally, in this clone two substitutions were found in the GFP sequence: His-232 to Leu, and Gly-233 to Cys.

The positive colonies selected for further analysis (clones 3, 5, 6, and 7) exhibited strong green fluorescence when irradiated with blue light (λₑₓ=460-480 nm). Fluorescent product content and GFP-P expression levels were determined in individual clones and compared after normalizing by protein content in bacterial lysates. The data is provided in Table 1. The apparent molecular masses of the proteins, as determined by SDS-PAGE, are reported in kilodaltons; fluorescence intensity, reported in AU/mg, was measured at λₑₓ 475/λₑₘ 508 nm at pH 8; GFP-P expression is reported as a percentage of total protein and was determined by densitometry of Coomassie-stained gels loaded with 10 μg of bacterial lysate.

TABLE 1
Properties of GFP Fusion Proteins

| | Apparent Mol. | fluorescence | GFP-P |
|---|---|---|---|
| Clone | Mass | intensity | Expression |
| 3, | 28.1 | 3410 | 5.8 |
| Hydrophilic | | | |
| 5, | 31.5 | 750 | 17.6 |
| Hydrophobic | | | |
| 6, N-Mutated | 29.8 | 1475 | 19.5 |
| Hydrophobic | | | |
| 7, Control, | 30.5 | 679 | 20.3 |
| C-Truncated | | | |
| Empty BSKS | — | 23 | — |
| Vector | | | |

Clones were selected with high levels of GFP-P expression (>5% total protein) which could be detected in a detergent-extractable fraction. The “hydrophilic” GFP-P variant (clone 3) was more fluorescent than the “hydrophobic” proteins (5-fold higher normalized fluorescence intensity as measured by fluorescence spectroscopy), and was readily extractable by simple treatment of bacteria with the ultrasound in the absence of detergents, as was N-terminal deletion mutant (clone 6). The mass of GFP-P (clone 3) was substantially less than expected (28 kD vs. 31 kD); this product was present in lysates at substantially lower amounts than other GFP fusions. Conversely, GFP-P in clones 5 and 7 (with no “hydrophilic” peptide fusion) was expressed at higher levels and was markedly less fluorescent (Table 1).

The binding of oxotechnetate to bacterial lysate components was studied after separation of bacterial proteins using SDS-PAGE. The removal of persulfate-generated free radicals during electrophoresis in the presence of 10 mM thioglycolic acid was found to be essential for prevention of cysteine oxidation. The resulting electrophoresis gels were used to study the binding of oxotechnetate(V) (de Kieviet, W. J. Nuclear Med. 22, 703-709, 1981) to GFP-P by re-chelation from a complex with glucoheptanoic acid (Gluceptate kit).

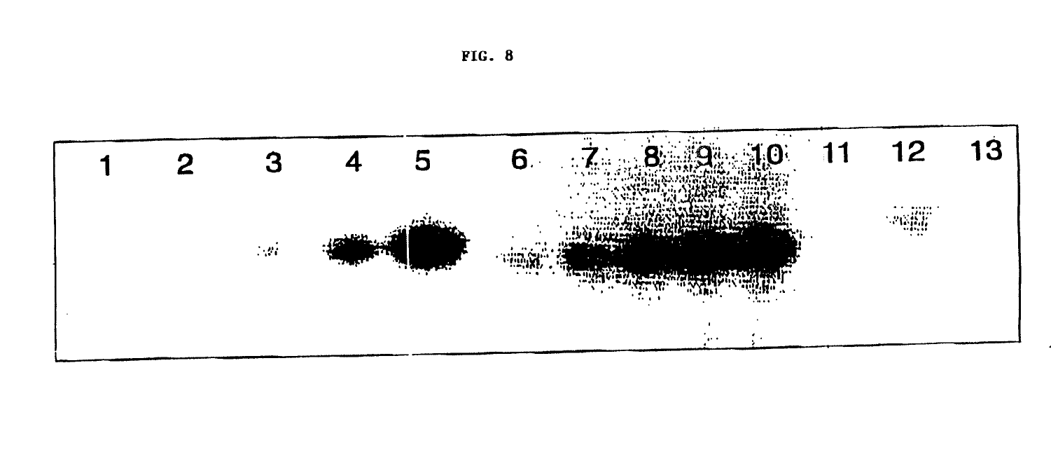

When a complex of oxotechnetate with glucaric acid was used instead, a considerable association of oxotechnetate with other major E. coli proteins, such as p50, was detected. The major oxotechnetate-binding component present was a 31 kD band (clone 5) or a 29 kD band (clone 6). The GFP-P products (clones 5 and 6) exhibited higher affinity for oxotechnetate than the truncated expression product of clone 7, as shown in FIG. 8.

FIG. 8 is a reproduction of the autoradiograph of the polyacrylamide gel described above (clone 3, lanes 1 (20 μg loading) and 2 (40 μg); clone 5, lanes 3 (10 μg), 4 (20 μg), and 5 (40 μg); clone 6, lanes 6 (5 μg), 7 (10 μg), 8 (15 μg), 9 (20 μg), and 10 (25 μg); clone 7, lanes 11 (20 μg) and 12 (40 μg); and BSKS, lane 13 (40 μg)).

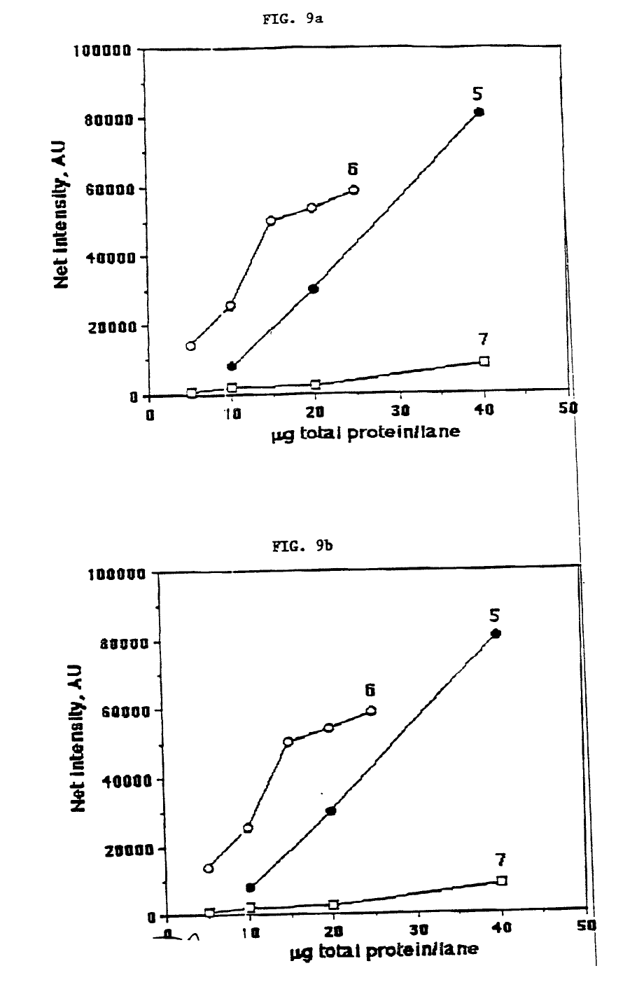

The bound radioactivity, expressed as net intensity of corresponding radiographic bands in FIGS. 9a and 9b (the numbers above the curves indicate the clone number), was normalized by protein content in individual bands, and corresponding values were compared.

There was a 10-fold difference in the normalized radioactivity bound to the GFP-P (305-31.5 kD bands) of the clones 5 and 7 (see FIGS. 8, 9a, and 9b). The specific activity of GFP-P labeling achieved for GFP-P isolated from clone 5 was 1.8 μCi/μg protein, with 10 μCi ^{99m}Tc glucoheptonate/ml used in each experiment. The binding of oxotechnetate was inhibited by 87% by a pre-treatment with N-ethyl maleimide in both GFP and GFP-P expression products, which confirms the involvement of thiol groups of cysteines in oxotechnetate association with GFP expression products. In experiments using “hydrophilic” GFP-P1 fusions, no detectable association of oxozechnetate with a corresponding protein band was detected, as shown in lanes 1 and 2 of FIG. 8.

To evaluate the potential of recombinant fusion proteins for oxotechnetate re-chelation from a complex with glucoheptanoic acid, several GFP (green fluorescent protein) fusions were constructed, which carried a C-terminal peptide with two GGGC repeats and expressed them in E. coli in phagemid vectors. GFP fusions were chosen to facilitate the isolation of transformed colonies and to obtain products with easily identifiable molecular mass, since protein lysates from E. coli transformed with empty BSKS vector yielded low levels of 30 kD protein expression. Thus, the interactions of “hydrophobic” and “hydrophilic” GFP C-terminal fusions with oxotechnetate were compared. “Hydrophilic” fusions were designed to create an electrostatic repulsion between GFP-P and glutathione disulfide.

All transformed clones displayed high fluorescence intensity of protein extracts, implying efficient expression of GFP-fusions. The two “hydrophobic” GFP-P2 variants studied were capable of re-chelation of oxotechnetate with high efficiency. The re-chelation was found to be inhibited by N-ethylmaleimide, which suggested that the re-chelation process was strictly dependent on the presence of cysteine thiol groups. In a separate control experiment involving a GGGC-free C-terminally truncated variant (clone 7), oxotechnetate re-chelation efficiency was shown to be lower, resulting in 10-fold less radioactivity associated with GFP-P (FIGS. 9a and 9 b).

No significant binding of oxotechnetate to the “hydrophilic” (i.e., glutamic acid-rich) C-terminal GFP-P1 fusions was demonstrated. This may have been due to the very high local negative charge of the C-terminus, which prevents proper coordination of oxotechnetium by the GGGC motif. Alternatively, since the GFP-P1 product appeared to have a smaller molecular mass than expected (28 kD vs. 30 kD), it may have been that this particular fusion protein may be susceptible to partial proteolysis with a loss of C-terminal peptide, in turn resulting in low content concentration of oxotechnetium-binding sites.

### Other Embodiments

It is to be understood that while the invention has been described in conjunction with the detailed description thereof, that the foregoing description is intended to illustrate and not limit the scope of the appended claims. Other aspects, advantages, and modifications are within the scope of the following claims.

For example, the new methods could also be used to image gene expression in transgenic animals. A transgene linked to an IMG can be injected (i.e., either directly or via any of the vehicles described above) into an embryo or the embryonic stem cells of an animal (e.g., a mouse or human). Since the RPC would potentially then be expressed in every cell of the animal, it is preferable that the RPCs used in this manner are non-toxic and either bind non-essential metals or form only short-lived complexes with the metal compounds.

What is claimed is:

1. A recombinant peptide chelate (RPC) comprising the structure:
(XGₐC)_{b}Z_{c}CG_{d}X;
wherein X and Z are any amino acid; G is glycine; C is cysteine; a is 1 to 4; b is 1 or 2; c is 0 to 4; and d is 1 to 4.

2. The recombinant peptide chelate of claim 1, wherein the (XGₐC)_{b}Z_{c}CG_{d}X structure is repeated one or more times.

3. The recombinant peptide chelate of claim 1, wherein c is 1 to 4, and each Z is selected independently from the group consisting of valine, proline, and glycine.

4. An imaging marker gene comprising a nucleic acid sequence that encodes a recombinant peptide chelate comprising the structure:
(XGₐC)_{b}Z_{c}CG_{d}X;
wherein X and Z are any amino acid; G is glycine; C is cysteine; a is 1 to 4; b is 1 or 2; c is 0 to 4; and d is 1 to 4.

5. A method of monitoring gene expression of a polypeptide in a host, the method comprising:
introducing into the host an expression vector comprising a nucleic acid sequence encoding the polypeptide and an imaging marker gene (IMG) encoding a recombinant peptide chelate (RPC) which chelates a metal compound;
administering to the host the metal compound in an amount sufficient to form RPC-metal complexes in the host; and
assaying for the RPC-metal complexes as an indication of expression of the polypeptide.

6. The method of claim 5, wherein the metal of the metal compound is a radioisotope.

7. The method of claim 5, wherein the metal compound is selected from the group consisting of ^{99m}TcO₄⁻, ^{99m}TcO²⁺, ^{188m}ReO²⁺, ^{99m}TcO³⁺, ^{188m}ReO³⁺, and compounds of Fe, Ga, In, and the lanthanides.

8. The method of claim 5, wherein the metal compound is initially chelated with a biocompatible ligand which is displaced by the recombinant peptide chelate.

9. The method of claim 5, wherein the recombinant peptide chelate comprises the structure:
(XGₐC)_{b}Z_{c}CG_{d}X;
wherein X and Z are any amino acid; G is glycine; C is cysteine; a is 1 to 4; b is 1 or 2; c is 0 to 4; and d is 1 to 4.

10. The method of claim 9, wherein the (XGₐC)_{b}Z_{c}CG_{d}X structure is repeated one or more times.

11. The method of claim 9, wherein c is 1 to 4 and each Z is selected independently from the group consisting of valine, proline, and glycine.

12. The method of claim 5, wherein the metal compound is a charged or electroneutral complex comprising the formula (O−Me(V))₁L, wherein Me(V) is selected from the group consisting of the gamma emitting isotopes of group VII transition metals, i=1 to 4, and L is selected from the group consisting of the mono- and di-saccharides.

13. The method of claim 12, wherein L is selected from the group consisting of saccharic acid, glucoheptonic acid, gluconic acid, glucuronic acid, glucooctanoic acid, sorbitol, glucosamine, mannitol, tartaric acid, citric acid, and malonic acid.

14. The method of claim 5, wherein the RPC-metal complexes are assayed by imaging the host using magnetic resonance imaging, magnetic resonance spectroscopy, planar scintigraphy, single photon emission tomography, positron emission tomography, or X-ray computed tomography.

15. The method of claim 5, wherein gene expression of the polypeptide is quantified by an imaging technique selected from the group consisting of magnetic resonance imaging, magnetic resonance spectroscopy, planar scintigraphy, single photon emission tomography, positron emission tomography, and X-ray computed tomography.

16. A system for measuring gene expression of a polypeptide in a host, the system comprising:
a metal compound, and an expression vector comprising a nucleic acid sequence encoding the polypeptide and an imaging marker gene (IMG) encoding a recombinant peptide chelate (RPC) which chelates the metal compound.

17. A system of claim 16, wherein the polypeptide is a therapeutic polypeptide.

18. A method of monitoring gene expression of claim 5, wherein the expression vector is prepared by
obtaining an imaging marker gene (IMG) encoding a recombinant peptide chelate (RPC) which chelates a metal compound; and
inserting the IMG into an expression vector comprising a nucleic acid sequence encoding the polypeptide.

19. A method of claim 5, wherein the polypeptide is a therapeutic polypeptide.
